(19) United States
(12) Patent Application Publication
(10) Pub. No.: US 2004/0087923 A1
(43) Pub. Date: May 6, 2004

(54) Core for an absorbent article and method for making the same

(76) Inventor: Robert Theodore Cole, Jackson, NJ (US)

Correspondence Address: RATNERPRESTIA, P O BOX 980, VALLEY FORGE, PA, 19482-0980, US

(21) Appl. No.: 10/284,876

(22) Filed: Oct. 31, 2002

(51) Int. Cl. A61F 13/20; A61F 13/15

(52) U.S. Cl. 604/365

(57) ABSTRACT

A core and method for making a core are provided for an absorbent article. The core includes a porous substrate configured to transfer liquid. A superabsorbent polymer is adhered to the porous substrate, and at least a portion of the superabsorbent polymer is pressed into the porous substrate. Additionally, an absorbent article and method for making an absorbent article are provided. The absorbent article includes a barrier configured to prevent the passage of liquid. A core is positioned adjacent the barrier, and includes a porous substrate. A superabsorbent polymer is adhered to the porous substrate, and at least a portion of the superabsorbent polymer is pressed into the porous substrate. A cover is positioned adjacent the core on a side of the core opposite from the barrier. The cover is configured to permit the passage of liquid to the core.

### FIELD OF THE INVENTION

This invention relates to cores used in a variety of absorbent products such as hygiene pads, disposable diapers, adult incontinence pads, and briefs, which products are required to absorb discharged bodily fluids. More particularly, this invention relates to improved absorbent articles and methods of making such articles.

### BACKGROUND OF THE INVENTION

Absorbent products typically include a core that comprises, among other components, a blend of cellulose pulp fibers and superabsorbent polymer, the latter material hereinafter being referred to as “SAP”. The pulp content of the core is usually 60-80 percent of the total core weight. The use of SAP in such a pulp-filled core is considered to be standard in the absorbent product industry. These polymers swell and form a gel when contacted with liquids. SAP's are capable of acquiring and retaining under pressure many times their weight in liquid. The polymers, usually in particulate or powder form, are typically disposed among pulp fibers to form cores to be used in products such as diapers, sanitary napkins, and absorbent mats.

Currently available absorbent articles may also include tissue and/or airlaid composites containing SAP particles and/or superabsorbent fibers. Traditional methods of absorbent article manufacture use SAP applied as a solid particulate material, typically distributed randomly over a tissue core or a cellulose composite. Although the SAP particles in such absorbent articles afford the advantage of providing very high water absorbency, many of the particles become dislodged and shake out during preparation of the articles on high speed manufacturing machines.

One attempt to deal with the issue of shakeout is described in U.S. Pat. No. 5,641,561 to Hansen et al. There are described absorbent composites made of fibrous material (e.g. cellulosic or synthetic material) and particulate superabsorbent polymers that are bound to the fiber via hydrogen bonding binder molecules. Superabsorbent polymer particles are mixed as particles with bleached kraft fluff, heated and spread out to dry. The binder adheres the SAP particles to the fibers. The dried product is then fed through a hammermill and shunted to an airlay line to produce a web containing 40% SAP particles attached to individual fibers.

U.S. Pat. No. 5,593,399 to Tanzer discloses an absorbent article, namely a diaper, having two layers attached to form pocket regions. Tanzer describes the use of SAP particles located within the pocket regions of the article to provide an absorbent laminate.

The use of hammermills requires substantial capital investment and, despite advances in the art, still tends to result in products where shakeout of the SAP particles is a problem. Beyond this, there is an ever-increasing demand for thinner and thinner absorbent articles that nonetheless have high fluid absorption capacity. Unfortunately, absorbent articles produced by the foregoing methods tend to be bulky, due to the fluffing effect of the air-layering process.

Pulp fibers in absorbent articles play a role in fluid retention and in containing the SAP particles. Additionally, the pulp fibers aid in wicking liquid to the SAP particles. Since the SAP's swell when wet, they collapse microscopic fluid-wicking channels between the pulp fibers. The result is a tendency for leakage to occur when a second flow of liquid is provided, at which time much of the SAP is inaccessible to the liquid due to this “gel-blocking” effect. Thus much of the SAP is inefficiently used.

Additionally, the use of cellulose pulp can slow the fluid acquisition rate when pulp fibers are compressed to form a compact absorbent article. Also, the use of cellulose pulp adds costs associated with operating a pulp mill on each and every absorbent product line. Pulp fibers also tend to make it difficult to provide an ultrathin product, since considerable bulk is required to produce an article of sufficient absorbency.

U.S. Pat. No. 5,938,650 to Baer et al. describes an absorbent core, free of wood pulp or other cellulosic materials. The core in Baer includes two thin outer porous layers which are receptive to body fluids. A quantity of SAP particles are loosely contained in individual unbonded open zones or pockets defined by a plurality of heat bond lines between the layers.

There remains a need for an improved core for an absorbent product that makes it possible to reduce or eliminate the use of pulp.

### SUMMARY OF THE INVENTION

In one exemplary embodiment, the invention provides a core for an absorbent article. The core includes a porous substrate configured to transfer liquid and an adhesive applied to at least a portion of the porous substrate. The core also includes a superabsorbent polymer (SAP) adhered by the adhesive to the porous substrate. At least a portion of said SAP is positioned between opposed surfaces of said porous substrate.

In another exemplary embodiment, the invention provides an absorbent article including a barrier configured to prevent the passage of liquid and a core positioned adjacent the barrier. The core includes a porous substrate configured to transfer liquid, an adhesive applied to at least a portion of the porous substrate, and a superabsorbent polymer (SAP) adhered by the adhesive to the porous substrate. At least a portion of the SAP is positioned between opposed surfaces of the porous substrate. The absorbent article also includes a cover positioned adjacent the core on a side of the core opposite from the barrier, wherein the cover is configured to permit the passage of liquid to the core.

In yet another exemplary embodiment, the invention provides a method of making a core for an absorbent article. The method includes the steps of applying an adhesive to at least a portion of a porous substrate and adhering a superabsorbent polymer (SAP) to the porous substrate with the adhesive.

In still another exemplary embodiment, the invention is a method of making an absorbent article. The method includes applying an adhesive to at least a portion of a porous substrate and adhering a superabsorbent polymer (SAP) to the porous substrate with the adhesive. The method also includes pressing at least a portion of the SAP into the porous substrate and between opposed surfaces of the porous substrate, thereby forming a core. Finally, the method includes interposing the core between a barrier and a cover.

According to still another exemplary embodiment, the invention provides a core for an absorbent article. The core includes a porous substrate configured to transfer liquid, wherein the porous substrate comprises a nonwoven material. The core also includes an adhesive applied to at least a portion of the porous substrate. A superabsorbent polymer (SAP) is adhered by the adhesive to the porous substrate, and at least a portion of the SAP is positioned between opposed surfaces of the porous substrate. The SAP is disposed in a loading gradient between the opposed surfaces such that a loading of the SAP at one of the opposed surfaces is substantially zero wt. %.

In yet another embodiment, this invention provides a method of making a core for an absorbent article including the steps of applying a superabsorbent polymer (SAP) to at least a portion of a porous substrate and pressing at least a portion of the SAP into the porous substrate and between opposed surfaces of the porous substrate.

### BRIEF DESCRIPTION OF THE DRAWINGS

FIG. 1 is a cross-sectional side view of an absorbent core, according to one exemplary embodiment of the invention.

FIG. 2 is a partial cross-sectional side view of an absorbent article employing an absorbent core such as that shown in FIG. 1, according to another exemplary embodiment of the invention.

FIG. 3 is a top view of the absorbent article of FIG. 2, showing optional elastic members 15.

FIG. 4 is a schematic diagram illustrating a process for making SAP-impregnated material suitable for preparing an absorbent core, according to a further exemplary embodiment of the invention.

FIG. 5 is a top view of a web inverter, such as may be used according to the invention in the process of FIG. 4.

FIG. 6 is an end view of a web of SAP-impregnated material passing over a first distribution plate suitable as an alternative means for distributing SAP according to the process of FIG. 4.

FIG. 7 is a top view of the web and distribution plate of FIG. 6.

FIG. 8 is an end view of a web of SAP-impregnated material passing over a second distribution plate suitable for use with the first distribution plate of FIG. 6.

FIG. 9 is a top view of the web and distribution plate of FIG. 8.

### DETAILED DESCRIPTION OF THE INVENTION

This invention will now be described with reference to specific embodiments selected for illustration in the drawings, wherein similar numbers indicate similar features. It will be appreciated that the spirit and scope of this invention are not limited to the embodiments selected for illustration. Instead, the scope of this invention is defined separately in the appended claims. Also, it will be appreciated that the drawings are not rendered to any particular proportion or scale.

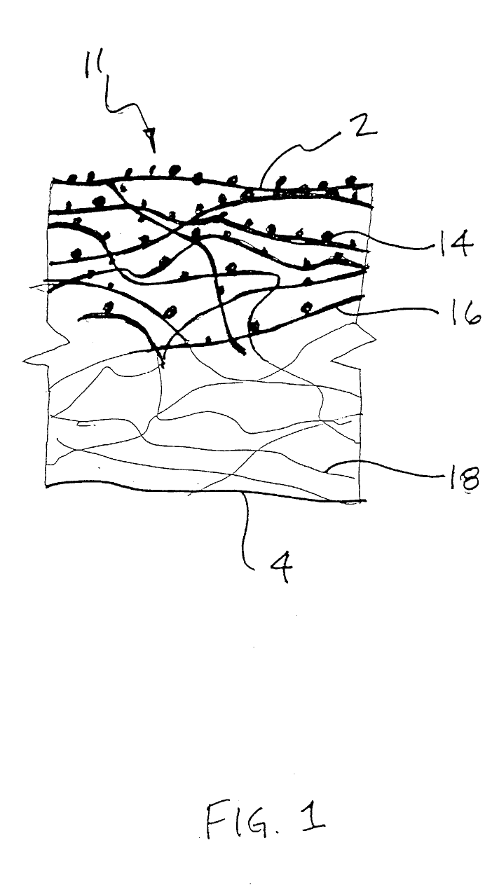

This invention provides an improved core for an absorbent product. Generally, with reference to FIGS. 1 through 4, a core 11 is provided for an absorbent article 10. The core 11 includes a porous substrate 18 configured to transfer liquid and having opposed surfaces 2 and 4 that may be generally parallel to one another. The opposed surfaces 2 and 4 define the thickness of the porous substrate 18.

SAP 14 is adhered to the porous substrate 18, and at least a portion of the SAP 14 is pressed into the porous substrate 18. By virtue of fixing SAP particles to the substrate 18, problems with shakeout of SAP are reduced. Moreover, since SAP particles 14 are fixed in a relatively open structure, there is still room for fluids to wick throughout the core even when the particles are swollen due to a previous exposure to fluid. Thus gel-blocking is reduced.

Additionally, an absorbent article 10 is provided, including a barrier 12 configured to prevent the passage of liquid. A core 11 is positioned adjacent the barrier 12, and includes a porous substrate 18. A SAP 14 is adhered to the porous substrate 18, and at least a portion of the SAP 14 is pressed into the porous substrate 18. A cover 20 is positioned adjacent the core 11 on a side of the core 11 opposite from the barrier 12. The cover 20 is configured to permit the passage of liquid to the core 11.

Moreover, this invention provides a method of making a core 11 for an absorbent article 10. The method includes adhering SAP 14 to a porous substrate 18, and pressing at least a portion of the SAP 14 into the porous substrate 18.

Furthermore, this invention provides a method of making an absorbent article 10. The method includes adhering SAP 14 to a porous substrate 18. At least a portion of the SAP 14 is pressed into the porous substrate 18, thereby forming a core 11. The core 11 is then interposed between a barrier 12 and a cover 20. In one embodiment, an ultrathin absorbent article is therefore provided which is made without the use of pulp fibers. Instead of a conventional core comprising pulp fibers with SAP's interspersed therein, the present invention makes it possible to provide an absorbent product formed in part from an acquisition or transfer layer coated with an adhesive to attach the SAP, thereby eliminating or reducing the need for pulp in the core. The elimination or reduction of pulp fibers from the core of the pad provides an ultra-thin product such as a bladder control pad. Typically, the thickness of an absorbent product is correlated to its absorbent capacity. That is, a typical pad or other absorbent product is thin if of low capacity, and thick if of high capacity. However, the present absorbent product provides a high capacity, ultra-thin structure.

FIG. 1 shows a cross-sectional side view of the structure of an absorbent core 11, according to one exemplary embodiment of the invention. Core 11 comprises a porous substrate 18, an adhesive 16, and a SAP 14. The adhesive 16 is shown as thick lines, indicating that it is attached to the surface of the fibers that constitute porous substrate 18. The loading of adhesive 16 and SAP 14 may be distributed in a gradient or layered manner through the thickness of core 11 as shown, in which case it may be possible for an absorbent article made from core 11 to be made either with or without a cover (to be described below). It will be appreciated that such gradients of adhesive 16 and SAP 14, if present, may not be identical, but may generally be interrelated. Alternatively, the distribution of adhesive 16 and/or SAP 14 may be substantially uniform through the thickness of core 11. This may for example be desirable when as heavy a loading of SAP 14 as possible is sought. In such a case, a cover may optionally be used in combination with the core 11.

If a gradient of SAP 14 such as shown in FIG. 1 is used, core 11 may be prepared in which SAP is concentrated on one side of the core, with little or no SAP on the opposing side. The result of this is the formation, in a single structure, of regions that perform respectively the functions of absorbency and liquid transfer. Thus the opposing side, having little or no SAP, may be placed nearer the user's skin, and perform the function of an acquisition-distribution layer. This may obviate the need for a separate acquisition-distribution layer, but it may also be used in combination with such a separate layer. It may also obviate the need for a cover, particularly if the portion touching the wearer's skin is made from porous substrate that is sufficiently hydrophilic and soft. For example, a carded multilayer unitized through-air porous substrate 18, wherein one side comprises low denier hydrophilic fibers, is available from PGI Nonwovens, Landisville, N.J. under the name TABICO™.

An exemplary porous substrate 18 is made from a non-woven material, which may be thermoplastic fibers or filaments, for example. Shape-retaining nonwoven fabrics are well known and are made by a variety of processes from fibers of polyolefins and polyesters. Where the fibers used are incapable of absorbing liquids, they may be treated with a surfactant for improved wettability. The material selected for the porous substrate 18 is preferably porous to allow rapid passage of liquid while sufficiently fine to embed the SAP 14 in a dry state. An example of one suitable material is heat bonded or point bonded nonwoven material comprising polypropylene fibers.

Other materials, which may contain other types of non-woven fibers, may be used for providing porous substrate 18. They may include for example a through-air bonded/carded web, a spun-bond bi-component non-woven web, and a web of cross-linked cellulosic fibers, apertured 3D film or the like. One particularly suitable material is a multi-denier nonwoven material available from PGI Nonwovens, Landisville, N.J., and has an overall basis weight of about 40 gsm, with high denier (about 10 denier) bi-component fibers situated on one surface and low denier (about 6 denier) bi-component fibers situated on the opposite surface. Such a multi-denier nonwoven, by virtue of having one surface that is relatively more porous, may faciltate the application of adhesive and the formation of a SAP gradient within the core. The bi-component fibers are made of a polypropylene inner core and polyethylene outer sheath. Preferably, the material used should be nonabsorbent and should permit the passage of liquid, but it may include hydrophilic fibers such as pulp within the interstices of the material.

Another exemplary porous substrate 18 may be formed from a liquid permeable film such as a 3-D apertured poly sheet comprising conical holes, available from Tredegar, located in Richmond, Va. Other substrate materials are contemplated as well.

Adhesive 16 is applied to the porous substrate 18 to encourage adhesion of the SAP 14 to the porous substrate 18. Typical adhesives are well known in the industry and are preferably water insoluble and rubber based.

According to an exemplary embodiment of the present invention, a SAP 14 is pressed into the porous substrate 18 so that at least a portion of the polymer particles are fixed in layer 18 to prevent migration or clumping of the particles 14. The porous substrate 18, containing the adhesive 16 and the SAP 14 pressed into it, forms the absorbent core 11.

The term SAP as used herein encompasses a hydrocolloid material, which is capable of absorbing many times its own weight of aqueous liquid. These materials are generally prepared by polymerizing one or more monomers, which if homopolymerized by conventional methods, would form water-soluble polymers. To render them water insoluble, these polymers or mixtures of them are typically crosslinked. Known polymers of this type are based on cross-linked salts of polyacrylic acid or polymethacrylic acid. Exemplary superabsorbent materials suitable for use include polyacrylamides, polyvinyl alcohol, ethylene maleic anhydride, and the like. Preferred are SAP's comprising crosslinked salts of polyacrylic acid. SAP 14 may be in the form of particulate matter such as for example powders, granules, fibers or fiber fragments.

The SAP 14 may have a relatively uniform particle size, or may have a distribution of particle sizes. A preferred form of SAP 14 is a granular or powdered material having a distribution of particle sizes ranging from about 45 μm to about 850 μm, preferably between about 106 μm and about 850 μm. The presence of some proportion of particles of small size may encourage effective penetration of such particles into the core 11, and may also increase the rate of liquid uptake when the absorbent article receives a liquid insult, due to the high surface area per unit weight of small particles.

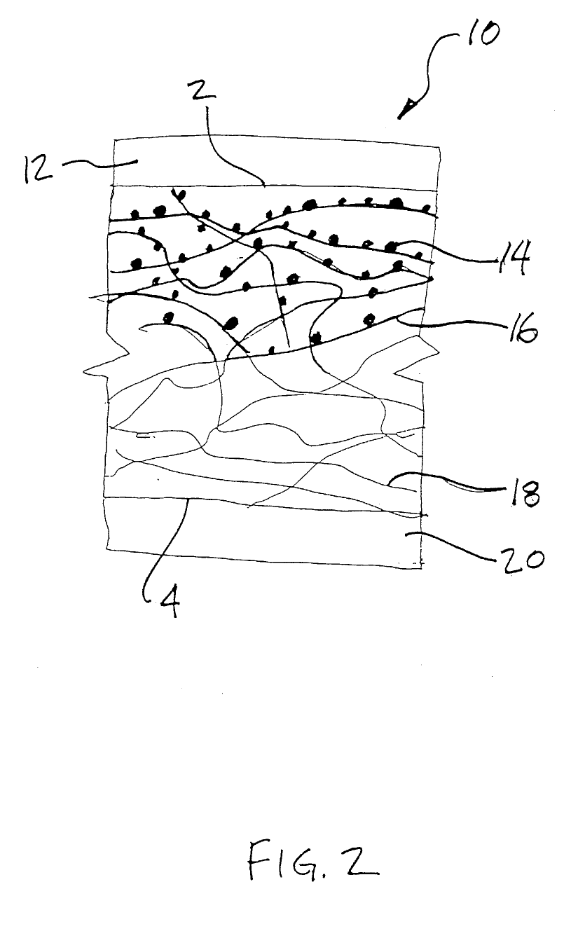

FIG. 2 shows a cross-sectional view of an absorbent article 10 according to one exemplary embodiment of the invention, employing a core 11 comprising an porous substrate 18 similar to that of FIG. 1 positioned adjacent a cover 20, with the side having more SAP 14 positioned adjacent a barrier 12. Cover 20 is liquid permeable and, when the article is in use, is in close proximity to the skin of the user. If used, the cover 20 is preferably compliant, soft feeling and non-irritating to the user's skin. Cover 20 can be made from any of the materials conventional for this type of use, for example spunbonded polypropylene or polyethylene, polyester, RAYON, Hydrofil® nylon fiber available from Allied Fibers, or the like. One suitable material is a hydrophilic 15.0-gsm spunbond polypropylene nonwoven from Avgol Nonwoven Industries, located in Holon, Israel. Another is a 17-gsm wettable nonwoven coverstock, made of thermal bond polypropylene, available from PGI Nonwovens, Landisville, N.J.

Other non-limiting examples of suitable materials that can be used as a cover 20 are woven and non-woven polyester, polypropylene, polyethylene, NYLON, and RAYON and formed thermoplastic films. Suitable films are described, for example, in U.S. Pat. No. 4,324,246 to Mullane and Smith and U.S. Pat. No. 4,342,314 to Radel and Thompson, both of which patents are incorporated herein by reference. Formed films may be selected for the cover 20 because they are permeable to liquids and yet non-absorbent. Thus, the surface of the formed film, which is in contact with the body, remains substantially dry and is more comfortable to the wearer.

If a cover 20 is used, its inner surface may be secured in contacting relation to the absorbent core. This contacting relationship results in liquid penetrating the cover 20 faster than if it were not in contact with the absorbent core. The cover 20 can be maintained in contact with the absorbent core by applying adhesive, optionally in spaced, limited areas, to an inner surface of the cover 20. Examples of suitable adhesives used for this purpose include the acrylic emulsion E-1833BT manufactured by. Rohm and Haas Company of Philadelphia, Pa. and acrylic emulsions manufactured by H. B. Fuller Company of St. Paul, Minn. Additionally, water-absorbing adhesives may be used, such as are known in the art. Also contemplated are thermoplastic hot melt adhesives such as 34-563A, available from National Starch, Inc.

Optionally, before a cover 20 is applied, a fluid distribution layer (not shown) may be attached adjacent and coextensive with the absorbent core, on the side of the core nearer the wearer, to improve distribution of bodily fluid more evenly over the full width and length of the core. Such a layer serves to manage, transport, accommodate and/or direct high volumes and high flow rates of urine into the core. The fluid-distribution layer can be made from any of a number of materials known in the art, including for examples the fibrous materials described above for use in making porous substrate 18. The fluid-distribution layer may be adhesively secured in place by any suitable construction adhesive or hydrophilic adhesive, e.g. Cycloflex adhesive available from National Starch and Chemical, Bridgewater, N.J.

Materials suitable for use in forming barrier 12, which is configured to prevent the passage of liquid, are well known in the industry. Such materials include, for example, films such as polyethylene, polypropylene, and copolymers, as are known in the absorbent article art. Suitable materials may include for example a liquid-impermeable laminate comprising a soft nonwoven (cloth-like/hydrophobic) on the outside and fluid-impermeable film (low gauge poly) on the inside. An example of this is a poly laminate available from Clopay Plastic Products Company, Cincinnati, Ohio, which consists of 0.6 mil polyethylene film and 17 gsm SMS (spunbond/meltblown/spunbond) nonwoven. Another version is a poly laminate 9B-396 available from Pliant Corporation of Newport News, Va., which consists of 0.3 mil copolymer film and 14 gsm SBPP (spunbond polypropylene) nonwoven. However, other laminate variations may be used in various gages and basis weights. For instance, other polymers (polypropylene, olefins, polyester, co-extruded polymers, etc.) or coatings (adhesive, synthetic rubber, latex, polyurethane, etc.) can be used in place of the polyethylene film. Other material components (polypropylene, polyethylene, bi-component fibers, polyester, cotton, RAYON, NYLON, olefins, etc.) can be used in either woven or nonwoven (spunbond, thermal bond, through-air bond, etc.) construction in place of the SMS outer cover. The preferred fluid-impermeable film for the liquid-impermeable laminate is a breathable 0.8 mil polyethylene version, which contains calcium carbonate, available from Tredegar Film Products, Richmond, Va. This material allows water vapor to pass through it, but does not permit the liquid itself to pass through it.

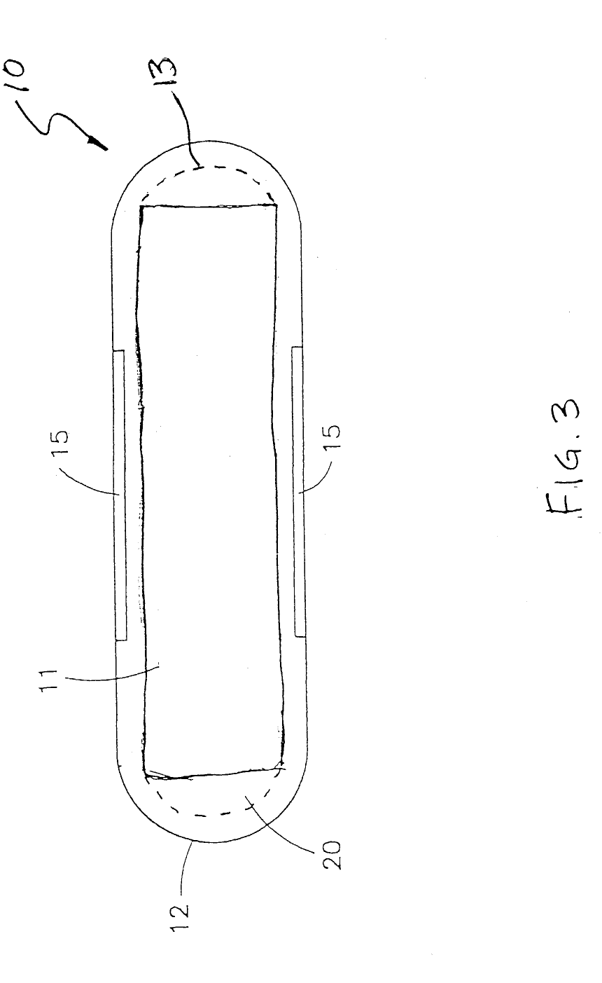

As shown in FIG. 3, a top view of an exemplary embodiment of an ultrathin absorbent product according to the present invention, the absorbent article 10 provides an ultrathin pad that includes an absorbent core 11 to receive bodily fluids. A cover 20 of the absorbent article 10 is positioned over the absorbent core 11, which cover 20 of the absorbent article 10 is intended to be positioned proximal to the user's skin. A barrier 12 is positioned on the opposite side of the absorbent core 11, and is provided to prevent the passage of any unabsorbed liquid to the outside of the absorbent article 10. The barrier 12 is the portion of the absorbent article 10 that is distal from the user's skin. The barrier 12 is preferably a liquid impermeable material such as a poly blend. The barrier 12 portion of the article 10 is proximal to, or in some embodiments attached to, clothing such as an undergarment in use. Optionally, patches of a positioning adhesive (not shown) may serve this securing function, with the positioning adhesive covered with a release paper that is removed prior to use. Such positioning adhesives and release papers are well known in the art. Additionally, in some embodiments of the present invention, elastic members 15 may be used to provide a curved pad for a comfortable, leak-resistant fit.

In the embodiment illustrated in FIGS. 2 and 3, the absorbent product 10 also comprises a barrier 12 that is preferably configured to prevent the passage of liquid. Barrier 12 is the back layer, or the layer that is positioned adjacent the absorbent core 11. The SAP 14 of the absorbent core 11 is located proximal to the barrier 12. This configuration permits liquid introduced into the absorbent article 10 to pass through the porous substrate 18, and to be absorbed by the SAP 14. The barrier 12 blocks passage of any unabsorbed liquid from the article 10 and provides support for the absorbent core 11.

Absorbent capacity of the present invention may be increased by having multiple absorbent cores 11 vertically stacked. In other words, two portions of a core (having a porous substrate, adhesive, and SAP) can be placed one on top of the other such that the resulting absorbent core includes multiple core thicknesses. In such a case, adhesive may be applied on both sides of the porous substrate 18 in order to provide adhesion between the layers of porous substrate.

The present invention also provides a method of making a core for an absorbent article such as absorbent article 10. One exemplary embodiment of the method includes the steps of adhering a SAP 14 to a porous substrate 18 and pressing at least a portion of the SAP 14 into the porous substrate 18.

FIG. 4 schematically illustrates an exemplary embodiment of the method of making SAP-impregnated material 46 suitable for preparing the absorbent core 11. The general method includes providing a moving web of porous substrate 18, which in FIG. 4 is moving from left to right, and sequentially delivering an adhesive 16 and of SAP 14 onto the surface of the web. Adhesive 16, which optionally may contain SAP particles, is applied to the porous substrate 18, by methods known in the art, such as by one or more spray nozzles 17 as shown in FIG. 4. Application of the adhesive 16 may be substantially even across and along the surface of porous substrate 18, or may be in patterns including but not limited to stripes, zigzags, dots, and circles. The adhesive may optionally be sprayed onto both the upper and lower surfaces of the web. The adhesive 16 may penetrate into the interstices of porous substrate 18, thereby providing sites where SAP 14 may be affixed to the matrix. In one exemplary embodiment, the spray nozzles 17 may be of a full cone or flat spray type, and may be fed by a pump. In addition, they may be nozzles, such as are known in the art, designed to afford a meltblown or spiral pattern of adhesive deposition. The nozzles 17 may be operated via an automatic computer-controlled system. However, manual operation of the spray nozzles is an additional option. Other adhesive application methods known in the art may be suitable as well, such as an automatically or manually fed adhesive roller applicator.

The total amount of adhesive 16 in core 11 may vary widely, depending on a multitude of factors including the amount of SAP 14 desired to be adhered, the coarseness and/or openness of porous substrate 18, the desired level of absorbent capacity, whether a separate cover is to be used in conjunction with the core, and possibly other considerations. In general, it will typically be desired to use enough adhesive that the SAP 14 is well adhered to the porous substrate 18, yet not so much as to create an impermeable barrier to fluid flow, such as might happen if for example surface pores in the substrate become closed up with adhesive and/or the entire substrate becomes permanently compacted upon going through the idle or calendering rolls (to be discussed below).

SAP 14, supplied from a drum or hopper 22, is then deposited onto the porous substrate 18 treated with adhesive 16 to form a combination layer 19. The SAP 14 may be introduced by direct application of the SAP 14 to the surface of the porous substrate 18. The drum 22 may be a receptacle with a funnel-shaped discharge port, as illustrated in FIG. 4, or may be any other dispensing means known in the art. When the drum 22 is in its opened position, superabsorbent polymer 14 is released from the drum funnel to provide a uniform deposition of the superabsorbent polymer 14 onto the porous substrate 18 containing the adhesive 16. At this point, some of the SAP 14 may fall into the interstitial spaces of porous substrate 18.

The discharged volume of superabsorbent polymer 14 from the drum is continuously replaced to ensure a steady application rate of the superabsorbent polymer 14. The regulation of the volume of superabsorbent polymer 14 in the drum 22 may be achieved via an automatic computer-controlled system. However, manual operation of the volume regulation is an additional option. Similarly, the operation between the opened and closed positions of the drum 22 is preferably automatic. However, manual operation between the opened and closed positions, via a manually operated switch for example, is an additional option. Other material application methods known in the art may be suitable as well, for example by use of a powder meter or an air injection powder sprayer.

The combination layer 19, which is the combination of the porous substrate 18 with the adhesive 16 and the SAP 14 deposited thereon, is then advanced through an “S” shaped path to encourage further penetration of the superabsorbent particles 14 into the porous substrate 18, and adhesion of the particles 14 to the porous substrate 18. As shown in FIG. 4, this may be achieved by the use of a pair of idle rolls 24 and 26. FIG. 4 shows an optional added pair of rolls 30 and 32, but any number may be used, and not necessarily in pairs.

As the combination layer 19 is advanced through the idle rolls 24 and 26, only a first portion of the SAP 14 will adhere to the porous substrate 18, and a second portion of the SAP 14 is not adhered. Distribution of SAP 14 across and into the porous substrate 18 may be encouraged by a cascading effect shown at 28, where unadhered SAP falls off of the surface of the moving web and then back onto the web and/or the idle rolls 26 and/or 32, thereby improving distribution of SAP across the web. After passing through the idle rolls, the web may pass through a web inverter 34 that turns the web upside down, whereupon still-unadhered SAP 14 may fall off and optionally be captured for recycle.

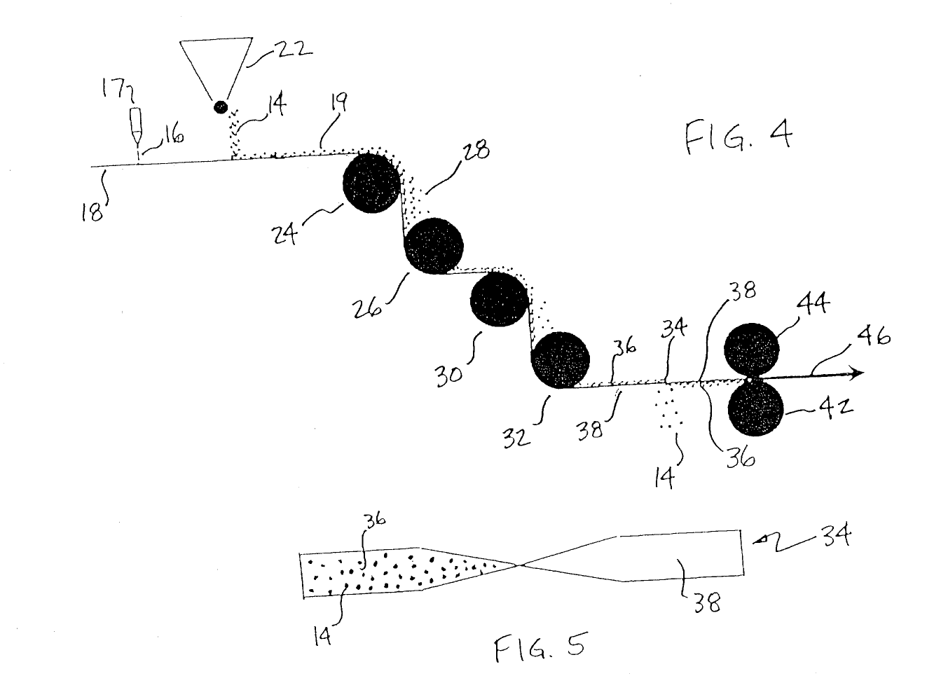

FIG. 5 shows a top view of web inverter 34, showing the SAP-treated surface 36 with SAP particles 14 on top upon entering the inverter and the untreated surface 38 on top after leaving. That is, the web is given a 180 degree twist in passing through web inverter 34.

The combination layer 19, after going through the web inverter 34, may be progressed through optional calender rolls 42 and 44 to press the SAP 14 into the porous substrate 18. This pressing step aids in the attachment of the superabsorbent polymers 14 to the porous substrate 18 containing the adhesive 16. The calender rolls 42 and 44 may provide a more even distribution of superabsorbent polymers 14 across the porous substrate 18, as well as additional penetration of SAP 14 into porous substrate 18, and adhesion to it, by what may be considered to be a kneading action which works the particles into the layer 18 and presses them into firmer contact with the fibers of layer 18, which in at least some locations have adhesive 16 on their surface.

The step of pressing the SAP 14 into porous substrate 18 may be achieved by other means than the calender rolls 42 and 44 shown in FIG. 4. For example, segments of material may be cut into manageable sizes, and the segments may then be pressed via a conventional pressing technique utilizing large flat plates. Virtually any means that applies pressure to press at least a portion of the SAP 14 into the porous substrate 18 may be used. The SAP-impregnated material 46 may then, if necessary, be dried to remove volatiles still present from the application of the adhesive 16, in the case where a solvent-based adhesive has been used. This may for example be done with an oven, or with a flow or air, or by other means known in the art. Typically however the adhesive will be a solventless adhesive, and no such step will be needed. The total amount of SAP 14 in core 11 may be from about 10 to about 90 wt. %.

An alternative exemplary embodiment of the invention is shown in FIG. 4A, in which the arrangement of the idle rolls 24 and 26 is such that at least some of SAP 14 on the surface of combination layer 19, which comprises porous substrate 18 treated with adhesive 16 dispensed from one or more spray jets 17, forms a cascade 28 of SAP off of the surface of combination layer 19 as is passes over idle roll 24. This cascading SAP subsequently falls back onto combination layer 19 after it exits idle roll 26, thereby enhancing distribution of SAP 14 across the porous substrate 18. Portions of SAP in cascade 28 that miss the surface of combination layer 19 may-optionally be caught in bin 47, and may optionally be recycled back into SAP-dispensing drum 22 for re-use, as indicated by arrow 21. Although a web inverter such as shown at 34 in FIG. 4 is not shown in the embodiment of FIG. 4A, it may optionally be used. As well, adhesive 16 may lie largely on the surface of porous substrate 18 as shown in FIG. 4A, or may penetrate into porous substrate. More than one pair of idle rolls may be used, although only one pair is shown in FIG. 4A.

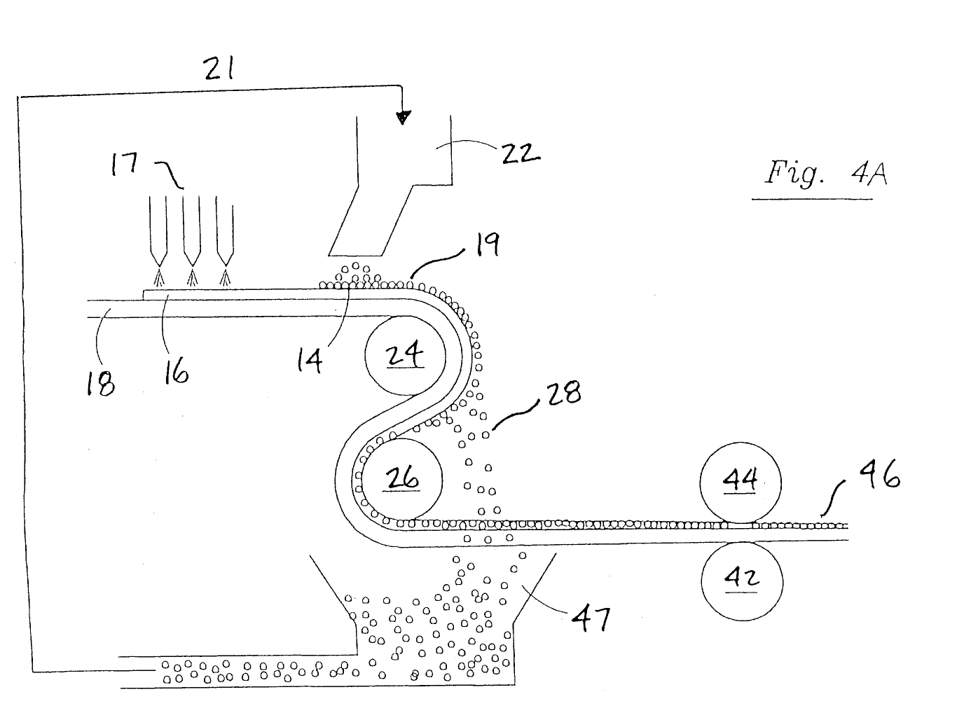

In yet another embodiment of the invention, shown in FIGS. 6-9, the distribution function performed by the idle rolls 24, 26, 30, and 32 in FIG. 4 may be performed by means of distribution plates 48 and 50, which are used in sequence, and which may be situated in place of the idle rolls in FIG. 4. FIG. 6 shows an end view of a first distribution plate 48, which lies lengthwise along the moving web of porous substrate 18 and causes the web to curve upward at the edges into a roughly U-shape, causing unadhered SAP 14 to cascade toward the middle of the web. FIG. 7 shows a top view of the web, which in this rendering is moving from left to right. The web is seen to exit the first distribution plate 48 with SAP 14 somewhat concentrated in the middle of the web.

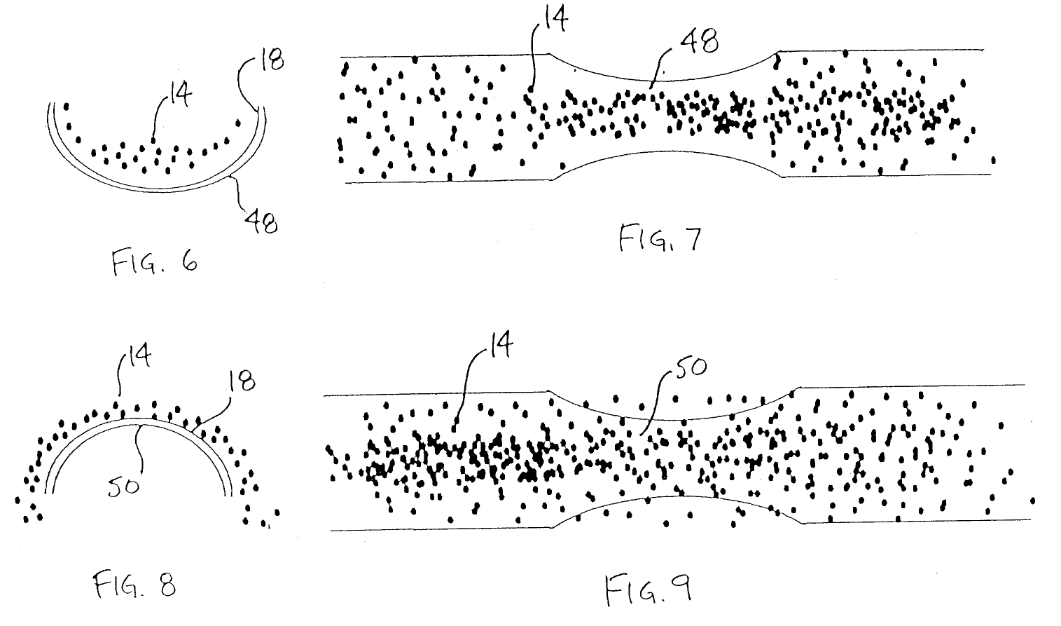

As shown in FIG. 8, the web may then pass over a second distribution plate 50, which lies lengthwise along the moving web of porous substrate 18 and causes the web to curve downward at the edges in roughly an inverted U-shape. As seen in FIG. 9, this may cause SAP 14 to migrate back toward the edges of the web, with some of it falling off and being recycled. The net result of passing across distribution plates 48 and 50 is thus to improve distribution. The sequence may be repeated, if desired, and may additionally be augmented with the use of vibrators or other devices (not shown) to encourage motion of SAP 14 across the surface of the web. Distribution plates as shown in FIGS. 6-9 may also be used in combination with idle rolls such as have been described in relation to FIGS. 4 and 4A above. Also, although the embodiment just described entails using an upright U-shaped plate followed by an inverted U-shaped plate, the opposite sequence may be used, or either may be used without the other.

From the foregoing discussion, it will be appreciated that the profile of SAP 14 as a function of depth in the SAP-impregnated material 46 is a function of a number of variables. These include, inter alia, the amount and particle size of the SAP 14 applied, the number and exact design of S-curves defined by idle rolls such as are shown at 24, 26, 30 and 32 in FIG. 4 (or at 24 and 26 in FIG. 4A), the pressures applied at these idle rolls, their speed, the exact shape and number of distribution plates such as shown at 48 and 50 in FIGS. 6-9, the speed and pressures at calender rolls 42 and 44, and the type, amount, and penetration depth profile of adhesive 16. Retention of SAP 14 in its intended location is the most important indicator of proper processing conditions.

The optimal depth profile of SAP 14 will be different for different applications, since it will affect total absorbency and fluid penetration time. Typically, but not exclusively, absorbent cores according to the invention for use as supplements to other absorbent structures will have deep penetration of SAP through the core. Conversely, in an absorbent article where a core according to the invention is the sole or primary absorbent structure, SAP will typically not penetrate more than about 50% of the way into the core 11, so that the substantially SAP-free surface of SAP-impregnated material 46 can perform its fluid distribution and acquisition functions unhindered.

In another exemplary embodiment, the invention further comprises cutting the SAP-impregnated material 46 to form an absorbent core 11 into a desired shape. For example, a rectangular shape with dimensions appropriate for a sanitary napkin or pad may be used. In other applications, appropriate shapes and dimensions may be selected as needed. Cutting the SAP-impregnated material 46 to form the absorbent core 11 may be accomplished through a variety of methods well known in the art, such as with the use of a blade.

In order to provide a complete absorbent article 10, the absorbent core 11 is then interposed between a barrier 12 and a cover 20. The core 11 comprises at least one layer of SAP-impregnated material 46, but may additionally comprise other layers, which may for example include one or more other layers of SAP-impregnated material. In one embodiment of the invention, two different SAP-impregnated materials 46 may be used, one of which is made from a softer, finer denier porous substrate material, and the other made from a coarser one. The layer made from the softer, finer denier material may be placed adjacent the wearer's skin, while the coarser layer may be placed adjacent the non-skin contacting surface of the finer layer, affording higher capacity. In such a configuration, it may if desired be possible to eliminate the use of a cover 20.

Core 11 may also or instead include pulp-containing layers (optionally containing SAP), and/or layers designed to enhance fluid distribution in and across the core 11, all of which are known in the art. The cover 20 is positioned adjacent to the absorbent core 11, proximal to the side of the porous substrate 18 that does not contain the SAP 14. Thus, the liquid that comes in contact with the cover 20 seeps through the cover 20 and the porous substrate 18. It is then absorbed by the SAP 14, which swells upon absorption and forms a gel. The barrier 12 inhibits the passage of any unabsorbed liquid through the article 10, thereby preventing accidental wetting or soiling of the wearer's clothing.

The method optionally includes applying elastic 15 to the absorbent article 10 to provide a curved pad or napkin, for example. Methods of applying elastic to sanitary napkins and the like are well known in the art, such as by the use of LYCRA® stretch fiber, available from DuPont. As explained by U.S. Pat. No. 5,593,399 to Tanzer et al., which is incorporated herein by reference, the elastic members 15 are secured to the absorbent article 10 in an elastically contractible condition, so that in a normal under-strain configuration, the elastic members 15 effectively contract against the absorbent article 10. The elastic members 15 can be secured in an elastically contractible condition in at least two ways. For example, the elastic members 15 may be stretched and secured while the absorbent article 10 is in an extended conformation, i.e. not bunched up or folded. Alternatively, the absorbent article 10 may be contracted, and the elastic members 15 secured and connected to the absorbent article 10 while the elastic members 15 are in their relaxed or un-stretched condition. Still other means, such as heat-shrink elastic material for example, may be used to gather the absorbent article 10.

In the embodiment illustrated in FIG. 3, leg elastic members 15 extend essentially along the complete length of the, crotch region of the absorbent article 10. Alternatively, elastic members 15 may extend the entire length of the absorbent article 10, or any other length suitable providing the arrangement of elastically contractible lines desired for the particular absorbent article 10 design. As shown in FIG. 3 at the dashed lines indicated at 13, core 11 may optionally have curved ends, and may in fact be of any convenient shape.

Elastic members 15 may have any of a multitude of configurations. For example, the width of the individual elastic members 15 may be varied. The elastic members 15 may comprise a single strand of elastic material, or may comprise several parallel or non-parallel strands of elastic material, or may be applied in a rectilinear or curvilinear arrangement. Where the strands are non-parallel, two or more of the strands may intersect or otherwise interconnect within the elastic member 15.

The elastic members 15 may be affixed to the diaper in any of several ways that are known in the art. For example, the elastic members 15 may be ultrasonically bonded, heat and pressure sealed using a variety of bonding patterns, or adhesively bonded to the absorbent article 10 with sprayed or swirled patterns of hotmelt adhesive. The various configurations of the inventions may have the elastic members located on the inner surface of the cover 20, next to the user's skin. Alternatively, the elastic members 15 may be interposed between the cover 20 and the barrier 12.

Absorbent articles according to the invention may be used in a variety of absorbent articles, including for example diapers, adult incontinence pads, and feminine hygiene products. Cores made according to the invention may constitute the sole or primary means of fluid absorption in an absorbent article, or may be used to augment other fluid absorption structures. They may be placed anywhere in an absorbent article, for example in locations where additional absorbency is desired.

Although this invention has been illustrated and described with reference to selected exemplary embodiments and modifications thereof, it will be appreciated that this invention is not limited to the exemplary embodiments selected for illustration. Also, additional variations to the illustrated embodiments may be made without departing from the spirit or scope of this invention.

What is claimed:

1. A core for an absorbent article, the core comprising:
a porous substrate configured to transfer liquid;
an adhesive applied to at least a portion of said porous substrate; and
a superabsorbent polymer (SAP) adhered by said adhesive to said porous substrate, at least a portion of said SAP being positioned between opposed surfaces of said porous substrate.

2. The core of claim 1, said porous substrate comprising a nonwoven material.

3. The core of claim 2 wherein said nonwoven material is a multidenier nonwoven material.

4. The core of claim 1, wherein said adhesive is applied to said porous substrate in a pattern.

5. The core of claim 1, wherein said SAP is disposed in a loading gradient between said opposed surfaces of said porous substrate, and wherein a loading of said SAP at a plane at or between said opposed surfaces is substantially zero wt. %.

6. The core of claim 5, wherein substantially all of said SAP resides in a portion of said porous substrate corresponding to less than about 50% of the thickness defined by said opposed surfaces of said porous substrate.

7. The core of claim 6, wherein a remaining portion of said porous substrate is substantially free of SAP.

8. The core of claim 1, wherein said SAP is selected from the group consisting of powder, particulates and fibers.

9. The core of claim 1, further comprising a plurality of porous substrates.

10. An absorbent article comprising:
a barrier configured to prevent the passage of liquid;
a core positioned adjacent said barrier, said core comprising a porous substrate configured to transfer liquid, an adhesive applied to at least a portion of said porous substrate, and a superabsorbent polymer (SAP) adhered by said adhesive to said porous substrate, at least a portion of said SAP being positioned between opposed surfaces of said porous substrate; and
a cover positioned adjacent said core on a side of said core opposite from said barrier, said cover being configured to permit the passage of liquid to said core.

11. The absorbent article of claim 10, said cover comprising a nonwoven material.

12. A method of making a core for an absorbent article comprising the steps of:
applying an adhesive to at least a portion of a porous substrate; and
adhering a superabsorbent polymer (SAP) to the porous substrate with the adhesive.

13. The method of claim 12 wherein said porous substrate comprises a multidenier nonwoven material.

14. The method of claim 12 further comprising pressing at least a portion of the SAP into the porous substrate and between opposed surfaces of the porous substrate.

15. The method of claim 12, said step of adhering the SAP to the porous substrate comprising advancing the porous substrate along an “S” shaped path.

16. The method of claim 12, said step of adhering the SAP to the porous substrate comprising adhering a first portion of the SAP to the porous substrate and recycling a second portion of the SAP for adhering to the porous substrate.

17. The method of claim 14, said step of pressing comprising calendering at least a portion of the SAP into the porous substrate.

18. The method of claim 12, further comprising cutting the core into a predetermined shape.

19. A method of making an absorbent article comprising the steps of:
applying an adhesive to at least a portion of a porous substrate;
adhering a superabsorbent polymer (SAP) to the porous substrate with the adhesive;
pressing at least a portion of the SAP into the porous substrate and between opposed surfaces of the porous substrate, thereby forming a core; and
interposing the core between a barrier and a cover.

20. The method of claim 19, further comprising, after said pressing step, cutting the core, the barrier, and the cover into a predetermined shape.

21. The method of claim 19, further comprising attaching elastic along an edge of the absorbent article.

22. The method of claim 19, further comprising forming the core into two or more layers.

23. The method of claim 22, said step of forming the core into two or more layers comprising slitting the core to form multiple portions and layering the portions.

24. A core for an absorbent article, the core comprising:
a porous substrate configured to transfer liquid, wherein said porous substrate comprises a nonwoven material;
an adhesive applied to at least a portion of said porous substrate;
a superabsorbent polymer (SAP) adhered by said adhesive to said porous substrate, at least a portion of said SAP being positioned between opposed surfaces of said porous substrate, said SAP being disposed in a loading gradient between said opposed surfaces such that a loading of said SAP at one of said opposed surfaces is substantially zero wt. %.

25. A method of making a core for an absorbent article comprising the steps of:
applying a superabsorbent polymer (SAP) to at least a portion of a porous substrate;
pressing at least a portion of the SAP into the porous substrate and between opposed surfaces of the porous substrate, thereby forming a core.

26. The method of claim 25, wherein said applying step comprises positioning substantially all of the SAP in a portion of the porous substrate corresponding to less than about 50% of the thickness defined by the opposed surfaces of the porous substrate.
